(19) United States
(12) Patent Application Publication
(10) Pub. No.: US 2024/0041767 A1
(43) Pub. Date: Feb. 8, 2024

(54) Mitochondria-Enriched Extracellular Vesicles

(71) Applicant: Duquesne University of the Holy Spirit, Pittsburgh, PA (US)

(72) Inventor: Devika Soundara Manickam, Pittsburgh, PA (US)

(73) Assignee: Duquesne University of the Holy Spirit, Pittsburgh, PA (US)

(21) Appl. No.: 18/227,026

(22) Filed: Jul. 27, 2023

Related U.S. Application Data
(60) Provisional application No. 63/394,777, filed on Aug. 3, 2022.

(51) Int. Cl. A61K 9/127; A61P 9/10; A61K 35/33; A61K 35/30; C12N 5/077; C12N 5/079

(52) U.S. Cl. CPC A61K 9/127; A61P 9/10; A61K 35/33; A61K 35/30; C12N 5/0656; C12N 5/0618; C12N 2501/999

(57) ABSTRACT

The present invention is a composition, and a method for making and administering, a pharmaceutical in unit dosage form in which mitochondria-enriched extracellular vesicles are delivered, “as is” or with an optional suitable carrier, after increasing the mitochondria content of the vesicles by generating the vesicles from a human brain endothelial cell line in the presence of one or more suitable promoting agents. Such mitochondria-enriched vesicles are effective to treat tissues needing amelioration of mitochondrial dysfunction or boosting of mitochondrial function. The EVs intrinsically are able to cross the blood-brain barrier and thus are particularly well suited for treating compromised tissues of the brain in situ. as well as other tissues under stress and in need of treatment not limited to neurologic tissues, and are typically administered parenterally.

### CROSS-REFERENCE TO RELATED APPLICATION

This patent application claims priority to, and incorporates herein by reference, U.S. provisional patent application No. 63/394,777, filed 3 Aug. 2022.

### FIELD OF THE INVENTION

The invention pertains to delivering naturally-produced mitochondria, as a pharmaceutical agent, via mitochondria-enriched extracellular vesicles produced in vitro.

### BACKGROUND OF THE INVENTION

Extracellular vesicles (EVs) are naturally secreted by cells and play a role in intercellular communication. The innate ability of EVs to transfer their cargo, including nucleic acids, lipids, and proteins, makes them attractive as promising drug delivery systems. EVs are being developed as carriers for drugs such as RNA, proteins, and small-molecule drugs. EVs can be classified based on their size into small EVs (sEVs) and medium-to-large EVs (m/lEVs) that are also referred to as exosomes and ectosomes/microvesicles, respectively. sEVs are reported to contain mitochondrial DNA and mitochondrial proteins, and m/lEVs incorporate mitochondria during their biogenesis. We collectively refer to sEVs and m/lEVs as types of EVs as described herein.

Previous implementation of EVs as delivery vehicles for exogenous agents have included, without limitation, delivery of anti-inflammatory drugs, anti-neoplastic agents and so forth.

Separately from EVs, mitochondrial dysfunction is known to play a causal role in a variety of acute and chronic diseases, such as type II diabetes, drug/toxin-induced liver injury and nephrotoxicity, cardiovascular and neurodegenerative diseases. Beyond these, other degeneration is suspected to be linked to, if not directly caused by, mitochondrial dysfunction, such as occurs in neurologic diseases such as stroke, Parkinson's Disease, Alzheimer's Disease, Amyotrophic Lateral Sclerosis (ALS) and Huntington's Disease—diseases or degenerations which also currently do not have satisfactory treatments by any means. For example, in the case of a stroke, there is an optimal time window of around three or four hours within which clot-dissolving treatments will beneficially be used to prevent further brain damage, by breaking open the clot that caused the problem in the first place. Even when clots can be dissolved, however, the action of dissolving the blood clot does not provide any actual benefit to the brain cells or neurons themselves—the treatment is more of a matter of disrupting harm, not providing improvement. Even apart from neurological diseases, there are other conditions that involve similar tissue stress that require treatment as much as neurological conditions do—such as lung, kidney or liver damage from various active agent toxicities or other insults. Important cancer drugs, for example, can cause (for example) noninfectious pneumonitis and other compromise of various organs, for which rejuvenating treatment would be very valuable if available.

Accordingly, a need remains for a way to combat mitochondrial dysfunction—or beneficially to boost mitochondrial function—in a way that is effective and saf.

### SUMMARY OF THE INVENTION

In order to meet this need, the present invention intrinsically enriches EVs with mitochondria for the purposes of delivering EVs to affected tissues needing amelioration of mitochondrial dysfunction or boosting of mitochondrial function. The EVs intrinsically are able to cross the blood-brain barrier and thus are well suited for treating compromised tissues of the brain in situ. Preparation of such EVs is accomplished by promoting, in vitro, the intrinsic creation of mitochondrially enriched EVs by stimulating a human brain endothelial cell line with one or more promoting agents such that the endothelial cells manufacture and excrete EVs having markedly increased quantities of mitochondrial load compared to EVs excreted without the presence of the promoting agent. In other words, the cell line makes the enriched EVs, and no mitochondria are otherwise exogenously added to the EVs. By “markedly increased” is meant about 10 to about 20 percent more mitochondrial content than EVs excreted without the presence of the promoting agent. Often, the promoting agent is a polyphenol. The current specific class of promoting agents is one or more of 5-aminoimidazole-4-carboxamide riboside; Resveratrol; N-[2-[3-(piperazin-1-ylmethyl)imidazo[2,1 b][1,3]thiazol-6-yl]phenyl]quinoxaline-2-carboxamide; Formoterol; 1-(2,5-dimethoxy4-iodophenyl)-2-aminopropane hydrochloride; N-[(3R)-3-(dimethylamino)-2,3,4,9-tetrahydro-1H-carbazol-6-yl]-4-fluorobenzamide; Rimonabant; Fenofibrate; Pioglitazone; Rosiglitazone; Dexamethazone; and Sildenafil. The human brain endothelial cell line used to manufacture and excrete the present mitochondrially enriched EVs are those human brain endothelial cell lines which are well known and commercially available. Although the invention embraces both mitochondria-enriched extracellular vesicles that are small (known small size) known as sEVs, the known medium-to-large extracellular vesicles (mitochondria enriched according to the invention) known as m/lEVs are preferred.

### DETAILED DESCRIPTION OF THE INVENTION

As described immediately above, the present invention intrinsically enriches EVs with mitochondria for the purposes of delivering EVs to affected tissues needing amelioration of mitochondrial dysfunction or boosting of mitochondrial function. Administration is typically if not always parenteral. The present EVs may be given alone or in conjunction with one or more suitable carriers or excipients which are non-reactive with the mitochondria-enriched extracellular vesicles. The EVs intrinsically are able to cross the blood-brain barrier and thus are well suited for treating compromised tissues of the brain in situ. Preparation of such EVs is accomplished by promoting, in vitro, the intrinsic creation of mitochondrially enriched EVs by stimulating a human brain endothelial cell line with one or more promoting agents, such that the endothelial cells manufacture and excrete EVs having markedly increased quantities of mitochondria (10-20% more) compared to EVs excreted without the presence of the promoting agent. In other words, the cell line makes the enriched EVs, and no mitochondria are otherwise exogenously added to the EVs. Often, the present promoting agent is a polyphenol. The current class of promoting agents is one or more of 5-aminoimidazole-4-carboxamide riboside; Resveratrol; N-[2-[3-(piperazin-1-ylmethyl)imidazo[2,1 b][1,3]thiazol-6-yl]phenyl]quinoxaline-2-carboxamide; Formoterol; 1-(2,5-dimethoxy4-iodophenyl)-2-aminopropane hydrochloride; N-[(3R)-3-(dimethylamino)-2,3,4,9-tetrahydro-1H-carbazol-6-yl]-4-fluorobenzamide; Rimonabant; Fenofibrate; Pioglitazone; Rosiglitazone; Dexamethazone; and Sildenafil. The human brain endothelial cell lines used to manufacture and excrete the present mitochondrially enriched EVs are those human brain endothelial cell lines which are well known and commercially available. While the invention embraces the mitochondria enrichment of any and all EVs created by brain endothelial cell lines, preferably the enriched EVs are the medium-to-large EVs (m/lEVs) rather than the small EVs (sEVs). Both m/lEVs and sEVs are known in the art heretofore; the innovation here is in intrinsically enriching their mitochondrial content by use of a promoter during EV generation.

As a summary of our current in-progress research validating the above, we noted that EVs were promising carriers for the delivery of biotherapeutic cargo such as RNA and proteins. We had previously demonstrated that the innate (unenriched) EV mitochondria in medium-to-large (m/lEVs), but not small extracellular vesicles (sEVs) can be transferred to recipient brain endothelial cultures and mouse brain slice neurons. We thus continued to confirm that the innate EV mitochondrial load can be further increased via increasing mitochondrial biogenesis in the donor cells, which is the essence of the invention described herein. We further describe now that transfer of mitochondria via EVs is an effective approach to rescue mitochondrial function in various damaged cells and tissues. For example, protection of mitochondrial integrity and function is a potent strategy to limit stroke-induced damage in Brain Endothelial Cells (BECs). Damaged mitochondria in BECs increases Blood Brain Barrier (BBB) permeability and worsens post-stroke outcomes. Importantly, BECs have a greater reliance on their mitochondrial load compared to peripheral/non-BBB endothelial cells—allowing the orchestrated maintenance of its structural integrity and metabolic function. We therefore describe herein the paradigm wherein supplementation of healthy mitochondria to BECs lining the damaged/ischemic BBB protect its structural and function and limit post-stroke damage—creating actual benefit and not just reduction of harm. Indeed, the present mitochondrially enriched EVs actually protect the BBB—by keeping exogenous substances “out of the picture” altogether, and delivering instead only the naturally, intrinsically produced mitochondria-enriched EVs. By avoiding exogenous substance introduction, a cascade of problems caused by introduction of foreign agents is avoided altogether. Moreover, the brain endothelial origin of the present mitochondria-enriched extracellular vesicles gives them an identity, by surface markers and otherwise, that makes them beneficially recognizable particularly by brain cells, when the present pharmaceuticals are used to treat neurological disorders and diseases affecting the brain.

Whereas the invention has been described broadly above, the following Examples are illustrative.

### Example 1

As described in greater detail below, we treated NIH/3T3, a fibroblast cell line and hCMEC/D3, a human brain endothelial cell (BEC) line using Resveratrol to activate peroxisome proliferator-activated receptor-gamma coactivator-1α (PGC-1α), the central mediator of mitochondrial biogenesis. Naïve EVs and mito-EVs isolated from the non-activated and activated donor cells were characterized using dynamic light scattering and nanoparticle tracking analysis. The effect of mito-EVs on resulting ATP levels in the recipient BECs were determined using Cell Titer Glo ATP assay. The uptake of Mitotracker Red-stained EVs into recipient BECs were studied using fluorescence microscopy and flow cytometry. In collecting the results, we noted that mito-m/lEVs—but not mito-sEVs—showed a larger particle diameter compared to their naïve EV counterparts from the non-activated cells, suggesting increased mitochondria incorporation. Mito-EVs were generated at higher particle concentrations compared to naïve EVs from non-activated cells. Mito-EVs increased the cellular ATP levels and transferred their mitochondrial load into the recipient BECs. We therefore concluded that the pharmacological modulation of mitochondrial biogenesis in the donor cells can change the mitochondrial load in the secreted m/lEVs. Mito-EVs remained functionally active at levels greater or at least comparable to naïve EVs.[011] We used two cell models: hCMEC/D3, a human BEC cell line and NIH/3T3 mouse fibroblasts as donor cells to activate PGC-1α via Resveratrol treatment. The rationale behind choosing two unrelated cell line models was to avoid any unintentional bias in studying the effects of Resveratrol. Secondly, our prior work showed that naïve EVs derived from donor BECs show increased uptake into the recipient BECs compared to EVs from a non-homologous, macrophage cell line. We wanted to determine if the BEC-derived mito-EVs retain the inherent targeting capabilities by comparing mito-EVs from fibroblasts vs. BECs. As described below, we report physicochemical characteristics of mito-EVs studied using dynamic light scattering and nanoparticle tracking analysis and the effects of mito-EV-treated recipient BECs.

Materials Resveratrol was purchased from Tokyo Chemical Industry (Portland, OR). Branched polyethyleneimine (PEI, 25 kDa), L-ascorbic acid, hydrocortisone, and human basic fibroblast growth factor were purchased from Sigma-Aldrich (Saint Louis, MO). Cell Titer Glo 2.0 reagent for ATP assay was purchased from Promega (Madison, WI) and Pierce BCA protein assay kits and MitoTracker Deep Red FM were procured from Thermo Scientific (Rockford, IL). Type I Collagen was purchased from Corning (Discovery Labware Inc, Bedford, MA), and endothelial cell basal medium-2 (EBM-2) was procured from Lonza (Walkersville, MD). Penicillin-Streptomycin solution and Chemically Defined Lipid Concentrate were procured from Invitrogen (Carlsbad, CA). Heat-inactivated fetal bovine serum was purchased from Hyclone Laboratories (Logan, UT). RIPA buffer was purchased from Alfa Aesar (Ward Hill, MA) and aprotinin was purchased from Fisher Bioreagents (Fair Lawn, NJ). Nitrocellulose membrane was purchased from GE Healthcare Life Sciences (Germany), and Intercept blocking buffer was purchased from LI-COR Inc. (Lincoln, NE). Polycarbonate centrifuge tubes were purchased from Beckman Coulter, Inc. (Brea, CA). Mouse monoclonal antibodies against ATPSA, GAPDH, and PGC1α were purchased from Abcam (Waltham, MA). Alexa Fluor 790-conjugated donkey anti-mouse and anti-rabbit IgG were received from Jackson ImmunoResearch Lab Inc (West Grove, PA).

Cell lines We used the human cerebral microvascular endothelial cell line (hCMEC/D3, Cedarlane Laboratories, Burlington, Ontario) between passage numbers (P) 25 and P35 as a brain endothelial cell model in all experiments. hCMEC/D3 cells were cultured in tissue culture flasks and well plates pre-coated with rat collagen I (0.15 mg/mL) in complete growth medium containing endothelial cell basal medium (EBM-2) supplemented with fetal bovine serum (5% FBS), penicillin (100 units/mL)-streptomycin (100 μg/mL) (pen/strep), hydrocortisone (1.4 μM), ascorbic acid (5 μg/mL), chemically defined lipid concentrate (0.01%), 10 mM HEPES (pH 7.4), and basic fibroblast growth factor (1 ng/mL). The cells were maintained in a humidified 5% CO₂ incubator at 37±0.5° C. (Isotemp, Thermo Fisher Scientific) and replenished with complete growth medium every 48 hours until they formed confluent monolayers. For sub-culturing and plating, hCMEC/D3 cells were washed using 1× phosphate buffer saline (PBS), detached with 1× TrypLE express (gibco, Denmark) followed by neutralization with complete growth medium. Cell suspensions stained with trypan blue (1:1 v/v ratio) were counted to calculate % live cells using a hemocytometer before passaging at 1:3 to 1:5 v/v ratios or plating in well plates at defined cell densities.

Mouse embryo fibroblast NIH/3T3 cell line (ATCC, Manassas, VA) was provided by Dr. Ellen S. Gawalt (Duquesne University, Pittsburgh, PA). Cells between P10 and P20 were used in all experiments. NIH/3T3 cells were grown in complete growth medium composed of modified DMEM/F12 supplemented with 10% FBS and 1% pen-strep solution. Culture medium was replaced with pre-warmed fresh medium every 48 h until cells reached 80-90% confluency. The cells were washed with 1× PBS, detached with TrypLE express, followed by neutralization with complete growth medium prior to passaging or plating.

Measurement of PGC-1α expression using western blotting (Activation of hCMEC/D3 brain endothelial cells and NIH/3T3 fibroblasts with Resveratrol and preparation of cell lysate for western blotting) hCMEC/D3 brain endothelial cells (BECs) and NIH/3T3 fibroblasts were seeded in a 24-well plate at 100,000 cells/well-seeding density in complete growth medium and cultured for 48 hours in a humidified incubator at 37° C. The cells were treated with different concentrations of Resveratrol ranging from 0.1 to 50 μM in complete growth medium for 24 h. Untreated cells were incubated with a complete growth medium and used as the control for the treatment groups. Post-treatment, the cells were washed with 1× PBS, and the cell suspension was collected in centrifuge tubes. The cell suspension was centrifuged at 300×g for 10 min at 4° C., washed with 1× PBS, and the cell pellet was lysed with 1× RIPA buffer containing 3 μg/mL aprotinin. The total protein concentration in the cell lysates was measured using a BCA assay.

Detection of PGC1α protein expression in hCMEC/D3 BECs and NIH/3T3 fibroblasts using western blotting A 40 μg cell lysate of untreated and Resveratrol treated hCMEC/D3 BECs and NIH/3T3 fibroblasts were mixed with 4× laemmli buffer and distilled water. The mixture was heated at 95° C. for 5 min using a heating block (Thermo Scientific). Denatured samples and standard molecular weight markers (ladder, 250-25 kD) were separated on a 4-10% gradient sodium dodecyl sulfate polyacrylamide gel at 120 V for 90 min using a PowerPac Basic setup (Bio-Rad Laboratories, Inc.). The gel was washed with deionized water for 30 min, and the proteins were transferred from the gel to a 0.45 μm nitrocellulose membrane using a transfer assembly (Bio-Rad Laboratories, Inc.) at 75 V and 300 mA for 90 min. Following that, the membrane was washed with tris-buffered saline pH 7.4 containing 1% tween 20 (T-TBS) for 45 min and blocked with Intercept blocking buffer (Li-COR Inc., Lincoln, NE) for an hour at room temperature. The membrane was incubated with rabbit polyclonal anti-PGCla antibody (1 μg/mL), and mouse monoclonal anti-GAPDH antibody (1 μg/mL) solutions in blocking buffer at 4° C. overnight. The membrane was washed with T-TBS and incubated with Alexa Fluor 790-conjugated secondary antibodies (0.05 μg/mL) in blocking buffer for an hour at room temperature. The membrane was washed with T-TBS and scanned under 700 and 800-nm near-infrared channel using an Odyssey imager (LI-COR1Inc., Lincoln, NE) at intensity setting 5. The densitometry analysis was performed using Image studio software.

Isolation of naïve EVs and mito-EVs from hCMEC/D3 and NIH/3T3 cells sEVs and m/lEVs were isolated from the EV-conditioned medium of hCMEC/D3 BECs and NIH/3T3 fibroblasts using a differential ultracentrifugation method we have previously reported. Briefly, hCMEC/D3 BECs and NIH/3T3 fibroblasts were cultured in 175 cm 2 tissue culture flasks (T175) until about 90% cell confluency. For naïve EVs, confluent cells were washed with pre-warmed 1× PBS and incubated with serum-free medium for 48 hours in a humidified 5% CO₂ incubator at 37±0.5° C. For isolating mito-EVs, the confluent cells were incubated with 10 μM Resveratrol in serum-free medium for 48 hours. Post-incubation, the EV-conditioned medium was collected in centrifuge tubes and spun at 300×g for 11 min and 2000×g for 22 min at 4° C. to pellet down apoptotic bodies and cell debris using an Eppendorf 5810 R 15 amp version centrifuge (Eppendorf, Germany). The supernatants were transferred into polycarbonate tubes to isolate naïve and mito-m/lEVs via centrifugation at 20,000×g for 45 min at 4° C. using a Sorvall MX 120+ micro-ultracentrifuge (Thermo Scientific, Santa Clara, CA). Next, the supernatant was filtered through 0.22 μm PES syringe filters, and the filtrate was centrifuged at 120,000×g for 70 min at 4° C. to collect naïve or mito-sEVs. Lastly, sEV and m/lEV pellets were washed with 1× PBS and suspended in either 1× PBS for particle diameter measurements and in vitro experiments or in 10 mM HEPES buffer pH 7.4 for zeta potential measurements. The total protein content in isolated sEVs and m/lEVs were measured using a MicroBCA assay kit. Briefly, sEVs and m/lEVs were lysed using 1× RIPA buffer at a 1:15 volume ratio. A 150 μL volume of EV lysates was transferred to a 96-well plate along with bovine serum albumin protein standards (0.5 to 200 μg/mL). An equal volume of the MicroBCA working reagent (reagent A: reagent B: reagent C at 25:24:1 volume ratio) was added to each well and the plate was incubated at 37° C. for 2 h. The absorbance was measured at 562 nm using a SYNERGY HTX multi-mode reader (BioTek Instruments Inc., Winooski, VT).

Dynamic light scattering (DLS) The particle diameter, dispersity indices, and surface charge of EVs were measured using dynamic light scattering. EV samples were diluted to a final concentration of 0.1 mg EV protein/mL in 1× PBS for particle diameter and in 10 mM HEPES buffer pH 7.4 for zeta potential measurements. The samples were analyzed using a Malvern Zetasizer Pro (Worcestershire, UK). All samples were run in triplicate. Average particle diameter, dispersity index, and zeta potential values were reported as mean±standard deviation.

Nanoparticle tracking analysis sEVs and m/lEVs were diluted at 1:100 and 1:200 ratios in 1× PBS and analyzed using multiple-laser Zetaview f-NTA Nanoparticle Tracking Analyzer (Particle Metrix Inc., Mebane, NC). Prior to measurement, ZetaView was calibrated with 100 nm polystyrene beads and EVs were analyzed at 520 nm to measure particle concentration. Average EV concentrations were reported as mean±standard deviation (n=6).

Uptake of Mitotracker-labeled naïve and mito-EVs into the recipient hCMEC/D3 cells (isolation of mitochondria-labeled naïve and mito-EVs) hCMEC/D3 BECs and NIH/3T3 fibroblasts were cultured in T175 flasks to confluency. The complete growth medium was removed and cells were washed with 1× PBS. Cells were treated with 100 nM MitoTracker deep red (MitoT-red) diluted in serum-free medium for 30 min in a 37° C. humidified incubator, and following that, the cells were washed with 1× PBS. For naïve EVs, cells were incubated with serum-free medium for 48 h, whereas for mito-EVs, cells were incubated with serum-free medium containing 10 μM Resveratrol for 48 h. Post-incubation, the conditioned medium was collected into polystyrene centrifuge tubes. MitoT-red-stained sEV (MitoT-red-sEV) and m/1EV (MitoT-red-m/lEV) were isolated from EV-conditioned media using the differential ultracentrifugation method described above. The EV protein content in MitoT-red-sEVs and MitoT-red-m/lEVs were determined using MicroBCA assay, and the samples were stored at −80° C. until further use.

Uptake of hCMEC/D3 BEC- and NIH/3T3 fibroblast-derived MitoT-red-EVs into recipient hCMEC/D3 cells using fluorescence microscopy hCMEC/D3 BECs (P30) were seeded in a 24-well plate at 100,000 cells/well-seeding density in complete growth medium and cultured for 48 hours in a humidified incubator at 37° C. The cells were treated with BEC-derived naïve and mito-MitoT-red-sEVs and MitoT-red-m/lEVs at 5, 10, and 25 μg EV protein/well in complete growth medium for 48 h in a humidified incubator. In addition, the cells were also treated with fibroblast-derived naïve and mito-MitoT-red-sEVs and MitoT-red-m/lEVs at 5 and 25 μg EV protein/well in complete growth medium for 48 h. Post-treatment, the cells were washed with 1× PBS, and incubated in phenol-red free growth medium. The cells were observed under an Olympus IX 73 epifluorescent microscope (Olympus, Pittsburgh, PA) using the Cyanine-5 (Cy5, excitation 651 nm, and emission 670 nm) and bright-field channels at 20× magnification. Images were acquired using CellSens Dimension software (Olympus, USA).

Uptake of hCMEC/D3 BEC- and NIH/3T3 fibroblast-derived MitoT-red-EVs into recipient hCMEC/D3 cells using flow cytometry hCMEC/D3 BECs (P31) were seeded in a 48-well plate at a seeding density of 50,000 cells/well in complete growth medium and cultured for 48 hours in a humidified incubator at 37° C. Unstained and untreated cells were used as a control, whereas cells stained with 100 nM MitoT-red for 30 min in complete growth medium were used as a positive control. The cells were treated with BEC-derived naïve and mito-MitoT-red-sEVs and MitoT-red-m/lEVs at 5, 10, and 25 μg EV protein/well in complete growth medium for 48 h in a humidified incubator. Post-treatment, the cells were washed with 1× PBS, dissociated using TrypLE Express, diluted with PBS, and collected into centrifuge tubes. For each sample, an aliquot of a 100 μL cell suspension was analyzed on an Attune N×T Flow cytometer and 10,000 events were recorded in FSC vs. SSC plots. The MitoT-red fluorescent signals were detected at 670/10-nm and percentage signal intensities were presented in histogram plots generated using Attune software version 3.2.3. Any MitoT-red background signals were gated using the control, untreated cells.

The effect of EVs on relative ATP levels in the recipient hCMEC/D3 BECs hCMEC/D3 BECs (P30-P33) were seeded in 96-well plates at a seeding density of 16,500 cells/well in complete growth medium and cultured for 48 h in a humidified incubator at 37° C. The cells were incubated in an oxygen-glucose deprived (OGD) medium (defined as 120 mM NaCl, 5.4 mM KCl, 1.8 mM CaCl₂, 0.8 mM MgCl₂, 25 mM Tris-HCl, pH 7.4) for 24 h in a chamber (Billups Rothenberg, Del Mar, CA) pre-flushed with 5% carbon dioxide, 5% hydrogen and 90% nitrogen at 37±0.5° C. simulating hypoxic conditions in vitro. For normoxic conditions, the cells were cultured in complete growth medium in a 37° C. humidified incubator. Post-OGD exposure, the medium was removed, washed with 1× PBS, and the recipient cells were incubated with naïve and mito-sEVs and m/lEVs at 1, 5, 10, and 25 μg EV protein/well for 24 h. Untreated cells were incubated with either OGD medium (for OGD control) or in complete growth medium (for normoxic control). Branched polyethyleneimine at 100 μg/mL in complete growth medium was used as a positive control. For intercellular ATP measurements, the treatment mixture was removed, cells were washed, and cells were incubated with a 1:1 v/v mixture of complete growth medium and Cell titer glo 2.0 reagent for 15 min at room temperature in the dark. Post-incubation, the cell lysates were transferred into a white opaque plate and relative luminescence units were measured at 1 s integration time using a SYNERGY HTX multi-mode reader (BioTek Instruments Inc., Winooski, VT). The relative ATP levels were measured by means known in the art.

Statistical analysis Statistical significance among the mean of controls and treatment groups or within treatment groups were analyzed using one-way analysis of variance (ANOVA) or two-way ANOVA at 95% confidence intervals using GraphPad Prism 9 (GraphPad Software, LLC). The notations for the different levels of significance are indicated as follows: *p<0.05, **p<0.01, ***p<0.001, ****p<0.0001.

Results and discussion Mitochondria are the primary source of cellular ATP and regulate important events such as ATP synthesis, cell differentiation, immune activation, and induction of apoptosis and mitophagy. Mitochondrial dysfunction is one of the major causes of cell death in various neurodegenerative disorders and brain injuries, including ischemic stroke. As cells cannot remain metabolically active without functional mitochondria, exogenous delivery of mitochondria or mitochondrial components is an effective approach to revitalize the damaged brain endothelial and neural cells in various CNS disorders. Our previous report demonstrated using transmission electron microscopy that in situ endothelial cellular buds (m/lEVs) contain mitochondria. In contrast, mitochondrial proteins such as ATP5A (a subunit of ATP synthase present in the inner mitochondrial membrane) and TOMM20 (translocase of the outer mitochondrial membrane receptor) were present in both sEVs and m/lEVs.

Resveratrol treatment increased PGC-1α protein expression in NIH/3T3 cells, but not in hCMEC/D3 cells Resveratrol-mediated modulation of PGC-1α protein expression in NIH/3T3 and hCMEC/D3 cells was determined using western blot analysis. NIH/3T3 and hCMEC/D3 cells was treated with Resveratrol at concentrations ranging from 0.1 to 50 μM for 24 h in complete culture medium to identify the optimal Resveratrol concentration that increases PGC-1α expression. In NIH/3T3 cells, Resveratrol treatment showed a concentration-dependent gradual increase in PGC-1α band densities, whereas band densities of GAPDH remained unchanged up to 25 μM. However, there was a considerable decrease in PGC-1α and GAPDH expression at 50 μM Resveratrol compared to untreated cells. The densitometry analysis of three independent western blots demonstrated that Resveratrol induced a concentration-dependent increase in PGC-1α expression, where 10 μM Resveratrol showed a statistically significant (p<0.01) increase in normalized PGC-1α expression compared to untreated cells. However, Resveratrol at 50 μM showed a considerable (p=0.07) decrease in PGC-1α expression, likely suggesting that the 50 μM concentration resulted in cellular toxicity.

On the other hand, hCMEC/D3 cells treated with Resveratrol at 0.1 and 1 μM for 24 h showed a slight increase in PGC-1α protein band densities compared to untreated cells. However, Resveratrol concentrations from 10 μM to 50 μM showed a gradual and considerable decrease in PGC-1α and GAPDH protein expression. The densitometry analysis of three independent western blots demonstrated that Resveratrol treatment up to 1 μM showed a modest 5% increase in normalized PGC-1α protein expression compared to untreated cells.

In conclusion, NIH/3T3 cells treated with 10 μM Resveratrol showed a significant increase in PGC-1α protein expression, a key activator of mitochondrial biogenesis.

Physicochemical characteristics of naïve EVs and mito-EVs using dynamic light scattering and nanoparticle tracking analysis We used dynamic light scattering to determine EV physicochemical characteristics such as particle diameters, dispersity indices, and surface charge. We compared these parameters in naïve—(from control, non-activated cells) and mito EVs-derived from Resveratrol treated NIH/3T3 and hCMEC/D3 cells. Naïve sEVs derived from NIH/3T3 showed an average particle diameter of 115 nm. In contrast, naïve m/lEVs showed an average size of 205 nm, confirming that m/lEVs are significantly (p<0.0001) larger than sEVs. Importantly, mito-sEVs (116 nm) did not show a statistically significant change in particle diameter compared to naïve sEVs. In contrast, mito-m/lEVs (280 nm) showed a statistically significant (p<0.0001) increase in particle diameter compared to naïve m/lEVs. The data substantiate that Resveratrol increases mitochondrial biogenesis due to PGC-1α activation as a result, the secreted mito-m/lEVs have a greater mitochondrial load compared to naïve m/lEVs. In addition, there were no statistical differences in the dispersity indices between naïve and mito-sEVs as well as naïve and mito-m/lEVs. Naïve sEV and m/lEV showed zeta potentials ranging from −20 to −25 mV. Interestingly, while mito-sEVs showed a significantly lower zeta potential, mito-m/lEVs showed a significantly greater zeta potential compared to their naïve EV counterparts. Consistent with the NIH/3T3-derived EVs, hCMEC/D3 derived naïve sEVs (125 nm) showed a significantly lower particle diameter compared to naïve m/lEVs (average 195 nm), confirming our prior observations. Mito-sEVs showed a lower particle diameter (95 nm, p<0.0001) compared to naïve sEVs. Importantly, hCMEC/D3-derived mito-m/lEVs (250 nm) showed a significantly (p<0.0001) greater particle size compared to naïve m/lEVs (195 nm). The increased particle diameters of mito-m/lEVs compared to the naïve m/lEVs were conserved regardless of the donor cell source: NIH/3T3 fibroblasts vs. hCMEC/D3 brain endothelial cells. The dispersity indices of naïve EVs and mito-EVs remained similar. hCMEC/D3-derived sEVs showed significantly lower dispersity indices than m/lEVs. Lastly, hCMEC/D3-derived naïve sEVs and m/lEVs showed zeta potential ranging from −20 to −23 mV. hCMEC/D3-derived mito-sEV showed a significantly (p<0.001) greater zeta potential compared to naïve sEVs, while no changes were noted between naïve and mito-m/lEVs.

We used nanoparticle tracking analysis (NTA) to determine if PGC-1α activation in the donor cells and the subsequent changes in the mitochondrial load affect the secretion of EVs as a function of its particle concentration. We measured the concentration of mito-EVs and naïve EVs using NTA. NTA measures the particle diameter and concentration based on the diffusion coefficient of individual particles captured in optical videos. The particle concentration of BEC-derived naïve sEVs and m/lEVs was about 4.0×10⁹ particles/mL, which was significantly higher than the fibroblast-derived naïve sEVs and m/EVs (1.65×10⁹ particles/mL). The data suggest that hCMEC/D3 BECs produced significantly (p<0.0001) more EVs compared to NIH/3T3 fibroblasts.

Importantly, Resveratrol treated hCMEC/D3 BECs released significantly (p<0.0001) increased mito-sEVs (6.0×10⁹ particles/mL) and mito-m/lEVs (1.2×10¹⁰ particles/mL) compared to non-treated BECs. Notably, mito-m/lEV concentration was significantly (p<0.0001) greater than mito-sEV, suggesting that Resveratrol-mediated activation of PGC-1α increases the mitochondrial load and as a consequence, the activated cells produce more EVs. It is likely that PGC-1α-mediated increase in cellular mitochondrial biogenesis accelerates mitochondria packaging into m/lEVs, which are subsequently released at a greater rate compared to naïve m/lEVs from non-activated donor cells. A similar trend was observed in Resveratrol-treated NIH/3T3 fibroblast-derived mito-EVs. The concentrations of mito-sEVs and mito-m/lEVs were significantly (p<0.0001) greater than the naïve EV counterparts. Notably, the concentration of fibroblast-derived mito-sEVs was comparable to BEC-derived mito sEVs, whereas the concentration of BEC-derived mito-m/lEVs was significantly higher than the fibroblast-derived mito-m/lEVs. Overall, Resveratrol-mediated PGC-1α activation significantly increased sEV and m/lEV concentrations regardless of the donor cell type, and the most increases were noted in the case of mito-m/lEVs. The PGC-1α-mediated mitochondrial biogenesis increases the content of mitochondrial DNA/proteins as well as mitochondria numbers in the cells that are eventually released into the sEVs and m/lEVs, respectively.

Mito-EVs show significantly greater mitochondria transfer compared to naïve EVs EVs contain mitochondrial components, including mitochondrial DNA, mitochondrial proteins and entire mitochondria. We compared the uptake of mito-EVs with naïve EVs in the recipient BECs to determine if the increased mitochondrial load in mito-EVs translate to a greater extent of uptake. Mito-EVs or naïve EVs were generated from cells pre-stained with MitoTracker Red that selectively stains polarized, functional mitochondria in addition to mitochondrial proteins. These labeled samples are referred to as MitoT-red-sEVs or MitoT-red-m/lEVs. Recipient hCMEC/D3 BECs were treated with naïve and mito-EVs at 5, 10, and 25 μg doses for 48 h, and EV-mediated mitochondrial transfer was interpreted as purple MitoT puncta under a fluorescence microscope.

Untreated cells did not show any MitoT-red associated signals indicating that only MitoT-red specific signals were detected under the Cy5 channel. Naïve and mito-sEV at 5 and 10 μg doses did not show positive signals in hCMEC/D3 cells for 48 h, however, MitoT-red-sEV at the 25 μg dose showed faint intracellular Cy5 signals suggesting low levels of mitochondrial transfer into the recipient BECs. Mito-sEV at 25 μg showed considerably higher MitoT-red signals compared to naïve sEV. Interestingly, MitoT-red-m/lEV at a dose as low as 5 μg showed intense intercellular purple puncta, suggesting efficient m/lEV uptake into the recipient BECs. The levels of m/lEV uptake increased substantially as the dose of MitoT-red-m/lEV increased from 5 to 25 μg. Notably, m/lEV-mediated mitochondria transfer was considerably higher than sEVs confirming our prior findings. The greater transfer of m/lEV mitochondria into the recipient BECs cells is likely due to a greater enrichment of functional mitochondria in m/lEVs compared to sEVs. Importantly, mito-m/lEVs at 5 to 25 μg doses showed considerably higher MitoT-red signals than their naïve m/lEV counterparts, also suggesting incorporation of a greater mitochondrial load in the mito-m/lEVs compared to naïve m/lEVs.

We also studied the transfer of fibroblast-derived EVs into the recipient BECs. Recipient BECs were treated with fibroblast-derived EVs at 5 and 25 μg doses for 48 hours. Consistent with the BEC-derived EVs, fibroblast-derived m/lEVs but not sEVs showed a greater mitochondrial load and consequent transfer into the recipient BECs. Importantly, mito-sEVs and mito-m/lEVs showed a considerably greater mitochondrial load and subsequent transfer into recipient BECs.

We also performed flow cytometry analysis to quantify EV-mediated mitochondrial transfer into the recipient BECs using MitoT-red-stained EVs. Recipient BECs were treated with BEC-derived naïve and mito-EVs at 5, 10, and 25 μg EV doses for 48 h, and the intensity of MitoT-red-EVs were analyzed using histogram plots. Untreated hCMEC/D3 cells were used as control and were gated for data analysis. Cells pre-stained with MitoT-red were used as a positive control and were about 77% MitoT+ve, suggesting the sensitivity to detect MitoT-red-stained polarized mitochondria and mitochondrial proteins. Naïve and mito-sEVs showed about 2 to 5% MitoT+ve cells. Importantly, 5 μg mito-sEV showed a significantly (p<0.001) greater mitochondrial transfer compared to naïve sEV, whereas no statistical differences were observed at higher doses of sEVs. Cells treated with m/lEVs at a low dose of 5 μg showed about 5% MitoT-positive signals, that increased by 2-fold at the 25 g EV dose suggesting efficient mitochondrial transfer into the recipient BECs. Importantly, mito-m/lEV showed significantly (p<0.01) greater mitochondrial transfer at 5 and 10 μg EV doses compared their naïve m/lEV counterparts. However, at a higher amount (25 μg EV protein), mito-m/lEV-mediated mitochondrial transfer was lower than naïve m/lEV.

Overall, mito-sEVs and mito-m/lEVs showed significantly greater mitochondrial transfer at lower EV doses compared to naïve sEVs and m/lEVs. We believe that the lack of difference between naïve and mito-EVs at the higher doses could be due to saturation of particle uptake into the recipient cells.

The effects of mito-EVs on relative ATP levels in the recipient hypoxic endothelial cells One of the main functions of mitochondria is ATP synthesis via oxidative phosphorylation. We measured the resulting relative ATP levels in the recipient BECs treated with naïve and mito-EVs-derived from hCMEC/D3 BECs and NIH/3T3 fibroblasts. The effects of naïve and mito-EV exposure on relative ATP levels in the hypoxic recipient hCMEC/D3 were measured using the Cell titer Glo ATP assay. First, hCMEC/D3 cells were treated with 1, 5, 10, and 25 μg of non-homologous, NIH/3T3 fibroblast-derived naïve and mito-EV for 24 h in oxygen-glucose deprived (OGD) medium. The relative ATP levels of untreated hypoxic cells were compared with cells cultured in normoxic conditions. OGD hCMEC/D3 showed about 40% ATP levels compared to normoxic cells, suggesting OGD mediated significant (p<0.0001) decrease in relative ATP levels. NIH/3T3-derived naïve sEV showed a significant (p<0.0001) increase in relative ATP levels in the recipient BECs that were treated at a low amount of one μg EV. Notably, the relative ATP levels increased from 120% at 1 μg naïve sEVs to 160% at 25 μg sEV, suggesting a dose-dependent increase in cellular ATP levels. Mito-sEVs also showed a significant (p<0.0001) increase in cellular ATP levels at all the doses compared to the OGD control. However, mito-sEV-mediated increase in ATP levels was significantly (at least p<0.01) lower than their naïve sEV counterparts. Furthermore, NIH/3T3-derived naïve and mito-m/lEV showed a significant (p<0.0001), three to five-fold increase in ATP levels compared to the untreated cells. Notably, the magnitude of mito-m/lEV-mediated increase in ATP levels in the cells treated at different EV doses was relatively similar compared to cells treated with naïve m/lEVs.

To evaluate the effect of EV source, the recipient BECs were treated at 1 to 25 μg EVs isolated from homologous hCMEC/D3 BECs as opposed to EVs derived from NIH/3T3 fibroblasts. Consistent with the observations noted in cells treated with fibroblast-derived EVs, BEC-derived sEVs showed a significant (p<0.0001) increase in ATP levels in the treated hCMEC/D3 cells compared to the control, untreated cells. Notably, BEC-derived naïve and mito-sEV showed a similar increase in ATP levels up to 5 μg EV dose. However, at 10 and 25 μg EV doses, mito-sEV-mediated increase in ATP levels was significantly lower than naïve sEV. Importantly, BEC-derived naïve and mito-m/lEVs also showed about a three to four-fold increase in relative ATP levels compared to the untreated cells. Interestingly, mito-m/lEV-mediated increase in ATP levels was significantly (p<0.01) higher than naïve m/lEVs. The observed increase in ATP levels of recipient BECs is likely due to m/lEV-mediated mitochondrial transfer into recipient cells that subsequently co-localized with the cell's mitochondrial network and participated in oxidative phosphorylation. Notably, only lower doses of BECs-derived mito-m/lEVs resulted in a significant increase in recipient cell ATP levels. These observations aligned with the results from the mito-EV uptake studies via fluorescence microscopy and flow cytometry. The data indicated that BEC-derived mito-m/lEVs effectively increased ATP levels compared to BEC-derived naïve m/lEVs and fibroblast-derived naïve and mito-EVs. These observations pointed to the selective effects of homologous BEC-derived EVs on the recipient BECs as opposed to the heterologous fibroblast-derived EVs on the recipient BECs. These findings aligned well with our previous findings on the effects of naïve BEC-vs. macrophage-derived EVs on the recipient BECs. These observations confirmed the inherent targeting capabilities of EV membranes to cells of the same origin.

### Example 2

Ischemic stroke causes brain endothelial cell (BEC) death and damages tight junction integrity of the blood-brain barrier (BBB). We harnessed the innate mitochondrial load of BEC-derived extracellular vesicles (EVs) and utilized mixtures of EV/exogenous 27 kDa heat shock protein (HSP27) as a one-two punch strategy to increase BEC survival (via EV mitochondria) and preserve their tight junction integrity (via HSP27 effects). We demonstrated that the medium-to-large (m/1EV) but not small EVs (sEV) transferred their mitochondrial load, that subsequently colocalized with the mitochondrial network of the recipient primary human BECs. Recipient BECs treated with m/lEVs showed increased relative ATP levels and mitochondrial function. To determine if the m/lEV-meditated increase in recipient BEC ATP levels was associated with m/lEV mitochondria, we isolated m/lEVs from donor BECs pre-treated with oligomycin A (OGM, mitochondria electron transport complex V inhibitor), referred to as OGM-m/lEVs. BECs treated with naïve m/lEVs showed a significant increase in ATP levels compared to untreated OGD cells, OGM-m/lEVs treated BECs showed a loss of ATP levels suggesting that the m/lEV-mediated increase in ATP levels is likely a function of their innate mitochondrial load. In contrast, sEV-mediated ATP increases were not affected by inhibition of mitochondrial function in the donor BECs. As substantiated below, intravenously administered m/lEVs showed a reduction in brain infarct sizes compared to vehicle-injected mice in a mouse middle cerebral artery occlusion model of ischemic stroke. We formulated binary mixtures of human recombinant HSP27 protein with EVs: EV/HSP27 and ternary mixtures of HSP27 and EVs with a cationic polymer, poly (ethylene glycol)-b-poly (diethyltriamine): (PEG-DET/HSP27)/EV. (PEG-DET/HSP27)/EV and EV/HSP27 mixtures decreased the paracellular permeability of small and large molecular mass fluorescent tracers in oxygen glucose-deprived primary human BECs. This one-two punch approach to increase BEC metabolic function and tight junction integrity may be a promising strategy for BBB protection and prevention of long-term neurological dysfunction post-ischemic stroke.

EVs are an emerging class of natural carriers for drug delivery due to their known roles in intercellular communication. They retain membrane signatures reminiscent of the donor cells from which they are derived and therefore possess inherent homing capabilities to recipient cells of the same type. They are also likely to be less immunogenic compared to synthetic nanoparticles. The smaller EVs (sEVs) range from 30 to 200 nm in particle diameter and their biogenesis involves the inward budding of endosomal membranes that transforms into multivesicular bodies followed by their fusion with the plasma membrane and sEV release into extracellular spaces. The biogenesis of medium-to-larger EVs (m/lEVs) involves their outward budding from the cell's plasma membrane with particle diameters ranging from 100 to 1000 nm. The selective packaging of the functional mitochondria and mitochondrial proteins in the m/lEVs motivated us to harness the m/lEV mitochondrial load as a therapeutic modality. Mitochondria play a central role in cellular energy production and regulation of cell death including apoptosis and autophagy [3]. Ischemia-induced mitochondrial dysfunction in the brain endothelial cells (BECs) lining the blood-brain barrier (BBB) initiates the generation of excessive reactive oxygen species, reduction in ATP levels, and consequently BEC death. Therefore, protection of mitochondrial function via exogenous mitochondria supplementation is a potent strategy to increase BEC survival post-ischemic stroke. Thus, we rationalize that mitochondria-containing m/lEVs derived from BECs can increase cellular bioenergetics and survival under hypoxic conditions.

Ischemic stroke-induced oxygen-glucose-deprivation (OGD) decreases ATP levels in the BECs leading to the accumulation of cellular cations and excitatory neurotransmitters. The cationic overload catalyzes enzymatic activities leading to generation of reactive oxygen species (ROS), impairment of mitochondrial ROS defense mechanisms, and the subsequent mitochondrial dysfunction. Therefore, restoring mitochondrial function is a viable strategy to reduce damage to the BECs. In addition, disruption of the BBB is a major hallmark of ischemic stroke that is associated with altered expression of tight junctions, adherens junction proteins, and BBB transporters. Early ischemia/reperfusion activates the polymerization of the actin cytoskeleton in endothelial cells which disassembles and internalizes the tight junction proteins and consequently lead to the loss of barrier properties of the BECs lining the BBB. Uncontrolled actin polymerization-induced breakdown of BBB leads to the infiltration of proinflammatory mediators, blood cells, circulatory immune cells, and toxins into the brain parenchyma, and leads to the secondary injury cascade. Hence, a combined strategy to decrease BEC death and their paracellular permeability, ultimately leading to protection of the BBB metabolic function and tight junction integrity is a potent approach for the treatment of ischemia/reperfusion injury.

Preclinical studies have demonstrated that endothelial, but not neuronal overexpression of heat shock protein 27 (HSP27), inhibited actin polymerization and elicited long-lasting protection against stroke-induced BBB disruption and neurological deficits. HSP27 binds to actin monomers and inhibits tight junctional protein translocation in endothelial cells. Intravenous administration of cell-penetrating transduction domain (TAT)-HSP27 rapidly enhanced HSP27 levels in brain microvessels, decreased infarct volumes, and attenuated ischemia/reperfusion-induced BBB disruption. Therefore, we selected human recombinant HSP27 protein as a therapeutic protein to determine whether EV/HSP27 mixtures may decrease tight junction permeability post-ischemia/reperfusion in culture conditions.

Administration of EV/exogenous HSP27 mixtures is a promising strategy that can allow harnessing the inherent targeting capabilities of the EVs to the recipient BECs along with the added benefits of their mitochondrial load. Here, we determine and test that innate mitochondria-containing BEC-derived EVs with exogenous HSP27 protein is a one-two punch approach to increase BEC survival and protect its tight junction barrier via decreasing the paracellular permeability post-ischemia. This approach will protect and strengthen the BBB that in turn can ameliorate long-term neurological damage and dysfunction. We tested the effects of adding a cationic copolymer, poly (ethylene glycol)-b-poly (diethyltriamine) (PEG-DET) to the EV/HSP27 mixtures to determine if the degree of EV/HSP27 interactions can be further improved. We have previously used PEG-DET polymer to form nanosized mixtures with superoxide dismutase protein and demonstrated a >50% reduction in brain infarct volume in a mouse model of acute ischemic stroke [15]. PEG-DET is a cationic diblock copolymer known for its safety and gene transfer efficacy in comparison to commercial transfection agents such as lipofectamine, polyethyleneimine, and other cationic polymers.

In this Example, we isolated sEVs and m/lEVs from hCMEC/D3: a human brain endothelial

cell line using a sequential ultracentrifugation method and characterized their particle diameter, zeta potential, and membrane integrity post-cold storage. According to recommendations from the 2018 Minimal Information for Studies of Extracellular Vesicles, EVs with an average particle diameter <200 nm should be referred to as small EVs (sEVs), and >200 nm as medium-to-large EVs (m/lEVs). In our studies, the average particle diameter of EVs that pelleted down at 120,000×g was about 122 nm, whereas the average diameter of EVs isolated at 20,000×g was about 185 nm. Therefore, we refer to EVs isolated at 120,000×g as sEVs, and EVs isolated at 20,000×g as m/lEVs. A mixture of sEV and m/lEVs at a 1:1 weight: weight ratio is referred to as EVs. While we collectively refer to both large (m/lEV) and small (sEV) vesicle fractions as EVs, we have studied the singular effects of both m/lEVs and sEVs. We showed the presence of mitochondrial components in m/lEVs using transmission electron microscopy and western blotting. We evaluated the effects of EV dose and incubation times on their uptake to the recipient BECs and demonstrated the colocalization of m/lEV-delivered mitochondria with the mitochondrial network of the recipient BECs. We studied the effects of EV exposure on the resulting relative ATP levels, mitochondrial respiration, and glycolytic capacity of the recipient BECs. We conducted a pilot experiment in a mouse middle cerebral artery occlusion model of stroke to determine its potential therapeutic effects and to determine if m/lEV treatment is safe from any adverse effects when administered intravenously (i.v.) to the mice. Twenty-four hours post-injection, we analyzed the brain infarct sizes of mice treated with m/lEVs or saline to determine its therapeutic effects. The physicochemical characteristics of EV/HSP27 binary mixtures and (PEG-DET/HSP27)/EV ternary mixtures were studied using native polyacrylamide gel electrophoresis and dynamic light scattering. The effects of EV/HSP27 on the paracellular permeability of small and large molecule fluorescent tracers were evaluated under ischemic and ischemia/reperfusion conditions in primary human BECs.

Materials. Recombinant human HSP27 was purchased from Novus Biologicals (Centennial, CO). Cell Titer Glo 2.0 reagent (ATP assay) was procured from Promega (Madison, WI). Micro BCA and Pierce BCA protein assay kits were purchased from Thermo Scientific (Rockford, IL). PEG-DET polymer was synthesized by aminolysis of PEG-poly(β-benzyl 1-aspartate) block copolymers with diethyltriamine as previously reported. Bio-Safe Coomassie G-250 stain was purchased from Bio-Rad Laboratories Inc.

EVs retained their physicochemical characteristics and membrane integrity upon revival from storage conditions. We collectively refer to sEVs and m/lEVs as EVs wherever applicable. We used dynamic light scattering to measure particle size and dispersity index of freshly-isolated EVs using a Malvern Zetasizer Pro (FIG. 1a). Average particle diameters of freshly isolated sEVs was 109.9±1.1 nm with a dispersity index (DI) of 0.39±0.02 and m/lEVs was 228.8±15.4 nm with DI of 0.35±0.03.

The study embodied in this Example aimed to evaluate the effects of the innate EV mitochondria and EV/HSP27 mixtures on the resulting metabolic function and tight junction integrity of ischemic BECs. This one-two-punch strategy allowed us to harness the innate EV mitochondria to increase cellular bioenergetics and mixtures of EV/exogenous HSP27 protein to reduce paracellular permeability in ischemic BECs. This novel approach to protecting the ischemic BECs can potentially limit damage to the BBB, an integral component. Ultimately, this one-two punch approach using EVs increased the BEC mitochondrial function due to the innate EV mitochondrial load, and EV/HSP27 protected tight junction integrity in ischemic BECs. Naïve m/lEVs and sEVs increased ATP levels (albeit m/lEV showed a greater magnitude of ATP increases), mitochondrial respiration, and glycolytic capacities in the recipient BECs. In this Example, i.v. injected m/lEVs resulted in data corroborating neuroprotection in a mouse model of ischemic stroke.

Although the invention has been described above with particular disclosure of specific constituents, methods, and results, the invention is only to be limited insofar as is set forth in the accompanying claims.

We claim:

1. A method for treating tissues under hypoxic or agent-induced stress, comprising the step of administering, in unit dosage form, one or more unit doses of a pharmaceutical composition containing mitochondria-enriched extracellular vesicles, wherein said mitochondria-enriched extracellular vesicles contain increased mitochondria therein due to their generation in the presence of a promoting agent.

2. The method according to claim 1 wherein said promoting agent is a polyphenol.

3. The method according to claim 1, wherein said promoting agent is selected from the group consisting of 5-aminoimidazole-4-carboxamide riboside, Resveratrol, N-[2-[3-(piperazin-1-ylmethyl)imidazo[2,1 b][1,3]thiazol-6-yl]phenyl]quinoxaline-2-carboxamide, Formoterol; 1-(2,5-dimethoxy4-iodophenyl)-2-aminopropane hydrochloride, N-[(3R)-3-(dimethylamino)-2,3,4,9-tetrahydro-1H-carbazol-6-yl]-4-fluorobenzamide, Rimonabant, Fenofibrate, Pioglitazone, Rosiglitazone, Dexamethazone, and Sildenafil.

4. The method according to claim 1, wherein said pharmaceutical composition is administered to treat hypoxic or agent-induced stress associated with Parkinson's Disease, Alzheimer's Disease, Huntington's Disease, Amyotrophic Lateral Sclerosis, stroke, type II diabetes, liver injury, nephrotoxicity, cardiovascular compromise; neurodegenerative diseases, lung tissue compromise, kidney tissue compromise, liver tissue compromise, brain tissue compromise, pancreas tissue compromise, gall bladder tissue compromise, bladder tissue compromise, and vascular tissue compromise.

5. The method according to claim 1, wherein as a result of generation of said mitochondria-enriched extracellular vesicles in the presence of said promoting agent, said mitochondria-enriched extracellular vesicles contain from about 10 to about 20% more intrinsically produced mitochondria than mitochondria produced in the absence of said promoting agent.

6. The method according to claim 1, wherein said pharmaceutical composition is administered to treat neurological disorders selected from the group consisting of Parkinson's Disease, Alzheimer's Disease, Huntington's Disease, Amyotrophic Lateral Sclerosis, and stroke.

7. The method according to claim 1, wherein said mitochondria-enriched extracellular vesicles are medium-to-large extracellular vesicles.

8. The method according to claim 1, wherein said unit dosage form contains at least one carrier or excipient.

9. A product-by-process composition, comprising a pharmaceutical composition in unit dosage form, wherein each dosage form contains a quantity of mitochondria-enriched extracellular vesicles containing enriched quantities of mitochondria therein due to their generation in the presence of a promoting agent and further wherein said promoting agent is selected from the group consisting of 5-aminoimi dazole-4-carboxamide riboside, Resveratrol, N-[2-[3-(piperazin-1-ylmethyl)imidazo[2,1 b] [1,3]thiazol-6-yl]phenyl]quinoxaline-2-carboxamide, Formoterol; 1-(2,5-dimethoxy4-iodophenyl)-2-aminopropane hydrochloride, N-[(3R)-3-(dimethylamino)-2,3,4,9-tetrahydro-1H-carbazol-6-yl]-4-fluorobenzamide, Rimonabant, Fenofibrate, Pioglitazone, Rosiglitazone, Dexamethazone, and Sildenafil.
